(19) United States
(12) Patent Application Publication
(10) Pub. No.: US 2008/0145856 A1
(43) Pub. Date: Jun. 19, 2008

(54) Method and Apparatus for Detecting A Ligand in A Fluid

(75) Inventor: Mirko Lehmann, Ebnat-Kappel (CH)

Correspondence Address: THE WEBB LAW FIRM, P.C., 700 KOPPERS BUILDING, 436 SEVENTH AVENUE, PITTSBURGH, PA, 15219, US

(73) Assignee: MICRONAS GMBH, Freiburg i.Br. (DE)

(21) Appl. No.: 11/951,779

(22) Filed: Dec. 6, 2007

(30) Foreign Application Priority Data
Dec. 8, 2006 (EP) 06025431

(51) Int. Cl. C12Q 1/68; G01N 21/76; B01J 19/00

(52) U.S. Cl. 435/6; 436/172; 422/68.1

(57) ABSTRACT

In a method for detecting at least one ligand that is suspected to be contained in the fluid that is being tested, at least one marker that is suitable for binding to the ligand is placed in the fluid. At least one measured value for the diffusion rate of the marker in the fluid is acquired. In addition, a reference value range for the diffusion rate exhibited by a complex consisting of at least the ligand and the marker that is bound to it in the fluid is provided. The measured value is compared with the reference value range and the presence of the ligand in the fluid is deduced depending on the results of this comparison.

The invention relates to a method for detecting at least one ligand that is suspected of being contained in a fluid that is to be tested, in which method at least one marker that is suitable for binding specifically to the ligand is placed in the fluid. The invention also relates to an apparatus for detecting at least one ligand that can be specifically marked with a marker and that is suspected of being present in the fluid being tested.

Such a method is disclosed in T. Vo-Dinh et al., Analytical Chemistry, Vol. 71, No. 2 of Jan. 15, 1999. In order to detect a ligand contained in a fluid, a receptor that is binding-specific for the ligand is immobilized on a nitrocellulose membrane at test sites that are arranged in the form of an array. The receptor is applied to the nitrocellulose membrane by means of a capillary needle, with the membrane being positioned under the capillary needle with the aid of a 2-axis positioning device. The receptors are then cross-linked with the nitrocellulose membrane either by exposure to UV light or by heat treating. The fluid that is to be tested is then brought into contact with the receptors in such a way that ligands contained in the fluid can bind to the receptors. The resulting receptor-ligand complexes are marked with an optical marker. Then any free marker that is present is rinsed off of the surface of the nitrocellulose membrane. In a further process step the nitrocellulose membrane with the optically marked receptor-ligand complexes located on it is positioned over a CCD 2-dimensional sensor and irradiated with an excitation radiation that excites the marker to emit a luminescence radiation, whose wavelength differs from that of the excitation radiation. The luminescence radiation is measured with the aid of the CCD 2-dimensional sensor. Based on the resulting measured values, the concentration of the ligand in the fluid is determined. In order to be able to use this method to test the fluid for the presence of as many different ligands as possible, it is advantageous to dispose as many test sites as possible on the nitrocellulose membrane on a predefined surface that roughly corresponds to the sensing surface of the CCD sensor. However, a problem arises, in that the test sites on the nitrocellulose membrane cannot be arranged as close as desired to each other since if the arrangement of the test sites is dense, there is a risk that when the receptors are applied to the test sites they will come into contact with receptors from an adjacent test site and mix together with them.

The object is therefore to provide a method and apparatus of the aforesaid type with which it is possible in a simple and easy way to detect a relatively large number of different ligands in the fluid.

This object is achieved when at least one measured value for the diffusion rate of the marker and/or of a complex, consisting at the least of the ligand and the marker bound to it, is acquired in the fluid, when a reference value range for the diffusion rate of the marker-ligand complex in the fluid is provided, and when the measured value is compared with the reference range and, depending on the result of this comparison, the presence of the ligand in the fluid is deduced.

The invention is based on the known principle that the diffusion rate of particles contained in a fluid decreases as the mass of the particles increases. Therefore, the diffusion rate of a marker bound to a ligand is less than that of an unbound marker. By measuring the diffusion rate of the marker and comparing the corresponding measured value with the reference value range, it is therefore possible to determine whether the marker is bound to a ligand and thus, whether the ligand is contained in the fluid. The marker preferably is selected in such a way that it is specifically bonded to the ligand, but not to any other molecules that might be contained in the fluid. In this method the binding of the marker to the ligand can also be accomplished indirectly by means of an additional particle, such as a detection antibody. The detection antibody can be added to the fluid separate from the marker and/or in the form of a detection antibody-marker complex.

In a preferred embodiment of the invention the marker is an optical marker, and the fluid is irradiated with excitation radiation, which excites the marker to emit a luminescence radiation, and the marker is detected on at least one optical 2-dimensional sensor that is sensitive to the luminescence radiation, and the diffusion rate of the marker is determined by measuring the velocity of the movement with which the image of the marker reproduced on the 2-dimensional sensor changes its position. The diffusion rate can also be measured optically in a simple manner. In this case the velocity of movement of the marker that is imaged on the 2-dimensional sensor can be determined using image processing methods that are known per se, for example with the aid of a microcomputer. In some cases it may even be possible in detecting the ligand in the fluid to utilize the distribution of the measured velocity values of the individual images of the marker on the optical sensor surface and to compare them with a reference distribution. The presence of the ligand can be deduced depending on the results of this comparison. Before the measured value for the diffusion rate of the marker-ligand complex is determined, any free markers that may still be present in the fluid must be removed from the 2-dimensional sensor detection zone. This can be done, for example, by binding the free markers to receptors that are immobilized outside of the 2-dimensional sensor detection zone on the surface of the solid, for example a defining wall of a measurement chamber containing the fluid. In the method, the binding of the free markers to the immobilized receptors can be promoted by reducing the temperature and/or inhibited or prevented by increasing the temperature.

The object of the invention referred to above can also be accomplished in a method in which the marker is an optical marker, the marker is selected in such a way that the spectrum of the luminescence radiation emitted by the marker depends on the binding of the marker to the ligand, the luminescence radiation emitted by the marker is measured for at least one wavelength and compared with a predetermined value range, and, depending on the results of this comparison, the presence of the ligand in the fluid is deduced.

A marker of this type, which operates on the principle of fluorescence resonance energy transfer, is disclosed in an article by John Bracht, “Fluorescense Resonance Energy Transfer,” ISCID Encyclopedia of Science and Philosophy 2006, International Society for Complexity, Information, and Design. The article can be downloaded from the Internet at the address <http://www.iscid.org/encyclopedia/Fluorescense_Resonance_Energy_Transfer>. The marker has two different fluorophores, which emit optical energy when excited at various wavelengths. One of the two fluorophores emits optical radiation when excited, which in turn stimulates the other fluorophore to emit optical radiation. The fluorescence resonance energy transfer is dependent on the distance between the two fluorophores, and this in turn is dependent on whether or not the marker is bound to the ligand. By measuring the wavelength of the optical radiation emitted by the marker and comparing this wavelength with the predetermined value range, it is therefore possible to determine whether the marker is bound to a ligand and thus, whether the ligand is contained in the fluid.

The invention is based on the knowledge that the diffusion rate of particles contained in a fluid decreases as the mass of the particles increases. Therefore, the diffusion rate of a marker that is bound to a ligand is less than that of an unbound marker. By measuring the diffusion rate of the marker and comparing the resulting measured value with the reference value range, it is therefore possible to determine whether the marker is bound to a ligand and therefore whether the ligand is contained in the fluid. The marker preferably is selected in such a way that it is specifically bonded to the ligand, but not to any other molecules that might be contained in the fluid. In this method the binding of the marker to the ligand can also be accomplished indirectly by means of an additional particle, such as a detection antibody. The detection antibody can be added to the fluid separate from the marker and/or in the form of a detection antibody-marker complex.

In a preferred embodiment of the invention the optical marker is preferably reproduced with the aid of imaging optics on at least two and preferably three optical 2-dimensional sensors that are sensitive to the luminescence radiation and whose planes of measurement preferably extended transversely to one another, and the movement velocities with which the images of the marker on the 2-dimensional sensor change their position are determined, which determines the diffusion rate of the marker from the movement velocities and the characteristic values for the relative position of the 2-dimensional sensors and preferably from the imaging optics. In this way, the diffusion rate of the marker can be determined three-dimensionally by means of triangulation, and the ligand therefore can be detected even more precisely in the fluid.

It is advantageous if a large number of the same markers are placed in the fluid, if the number of markers whose diffusion rate corresponds to the reference value range is determined, and if from this count the concentration of the marker in the fluid is determined. Thus the ligand can be determined both qualitatively and quantitatively with this method.

In a preferred embodiment of the invention the fluid is irradiated with a planar beam of excitation radiation whose plane of radiation preferably is parallel to the plane of the 2-dimensional sensor. in this way it is possible to investigate a particular layer of the fluid that is irradiated with the radiation beam for the presence of the ligand.

It is advantageous if, in order to obtain images on the 2-dimensional sensor of ligands located in various areas of the fluid, the radiation beam is moved and/or tipped transverse to the plane extending from the 2-dimensional sensor. The concentration of the ligand can then be measured for various locations in the fluid.

In a preferred embodiment of the invention at least two types of markers that are binding specific for various ligands and that emit various luminescence radiation wavelengths when irradiated with excitation radiation are placed in the fluid, and the diffusion rates for the individual marker types are determined by measuring the rates of movement with which the images of the marker of the given marker type change their position on the 2-dimensional sensor in each of the wavelengths and by comparing the resulting measured values for the diffusion rates in each case with a reference value range assigned to one of the respective marker types and, in each case depending on the result of the comparison, deducing the presence of the ligands that are binding-specific for the given marker type in the fluid. In this way, a number of ligands can be detected simultaneously in the fluid in a single test, and/or their concentration can be measured in the fluid.

It is particularly advantageous when at least the one ligand is prepared by separating a least one substrand from a DNA and/or RNA molecule present in the fluid, and replicating it by means of a polymerase chain reaction. The concentration of at least one replicated substrand can then be measured on-line, in order to check the progress of the chain reaction and to possibly control the progress, for example by heating and/or cooling fluid. The separation of the substrand can be accomplished by means of a primer added to the fluid. Is also possible to add a number of different primers to the fluid in order to separate and simultaneously copy different substrands from the DNA or RNA.

It is advantageous if the fluid is heated and/or cooled to a predetermined temperature before the measured value for the diffusion rate of the marker is determined and if a measured value for the diffusion rate is determined at least two different fluid temperatures. In this case it may be assumed that the diffusion rate of the marker or the marker-ligand complex increases as a temperature increases and that the ligand therefore is detected even more precisely in the fluid and that its concentration can possibly be determined if the diffusion rate is measured at different temperatures.

In a preferred embodiment of the invention at least two types of markers that are binding-specific for different ligands and that differ from each other with respect to the extinction time of their luminescence radiation are placed in the fluid, the radiation of the fluid with an excitation radiation is interrupted or ended and for the images of the individual markers on the 2-dimensional sensor in each case at least one measured value is determined for the extinction time, and the images are assigned to a marker type by comparing these measured values with reference values, and the diffusion rates for the individual marker types are determined by measuring the movement velocities with which the images of the marker of the given marker type change their position on the 2-dimensional sensor, and the resulting measured values for the diffusion rates are each compared with a reference value range assigned to the marker type and, depending on the result of the comparison, the presence of the ligand that is binding-specific for the respective marker type is deduced in the fluid. The individual images of the markers on the 2-dimensional sensor can therefore be assigned to a marker type based on their extinction time.

In a preferred embodiment of the invention, the individual markers have a different mass. The mass can be selected in such a way that the diffusion rate of the markers or the marker-ligand complexes lies in a range that is favorable for determining the measured value. The mass of the markers can be changed by appending molecules.

The object of the invention referred to above is achieved with respect to the apparatus in that the apparatus has a measuring device for measuring the diffusion rate that the marker and/or a complex, consisting at least of the ligand and the marker bound to it, exhibits in the fluid, that the apparatus has a reference value generator for providing a reference value range for the diffusion rate of the marker-ligand complex, and that the measurement device and the reference value generator are connected to a comparison device for the purpose of comparing the measured diffusion rate with the reference value range.

The presence of the ligand in the fluid can be deduced depending on the results of the comparison. If the measured diffusion rate coincides with the reference value range, the ligand is contained in the fluid. If the measured diffusion rate lies outside the reference value range, the ligand is not contained in the fluid. The reference value generator preferably is a data memory.

In a preferred embodiment of the invention the device has a receiving area for the fluid, and the measuring apparatus has at least one optical 2-dimensional sensor that is sensitive to a luminescence radiation emitted by the marker and that is disposed relative to the receiving area in such a way that a luminescence radiation emitted by a marker present in the receiving area is reproduced on the 2-dimensional sensor, and the 2-dimensional sensor is connected to an evaluation device, which is designed in such a way that it determines the diffusion rate from the changes in the measurement signal of the 2-dimensional sensor over time. In this way the marker can be excited by means of an excitation radiation and/or a chemical reaction to emit the luminescence radiation. An imaging optical system can be disposed between the 2-dimensional sensor and the receiving area. The diffusion rate can be determined in the evaluation device with the aid of image processing methods that are known per se by determining the velocity of movement with which the image of the marker moves on the 2-dimensional sensor.

The apparatus preferably has at least three of the optical 2-dimensional sensors, and the planes of extension or the optical axes of these 2-dimensional sensors are arranged transverse to each other in order to measure the diffusion rate in three dimensions. In this way the diffusion rate can be measured by means of vector calculations.

The receiving area for the fluid preferably is enclosed by a measurement chamber, whereby at least one 2-dimensional sensor and/or at least one light source for sending an excitation radiation for the marker is (are) integrated into a wall of the measurement chamber. In this way the device can have compact dimensions. The measurement chamber can be a flow-through chamber (flow cell) or an open measurement chamber, such as a trough.

It is advantageous if the measurement apparatus has at least two 2-dimensional sensors that are sensitive for different spectral ranges and/or if optical filters, which allow optical radiation to pass in different spectral ranges, are placed ahead of them. It is possible to simultaneously determine the diffusion rate of a number of ligands that are marked by markers that emit luminescence radiation in different spectral ranges.

Is desirable if the apparatus has a temperature control system for the receiving area. This can be integrated into one of the measurement chamber walls.

Examples of embodiments of the invention are explained in greater detail below based on the drawing. The drawing shows:

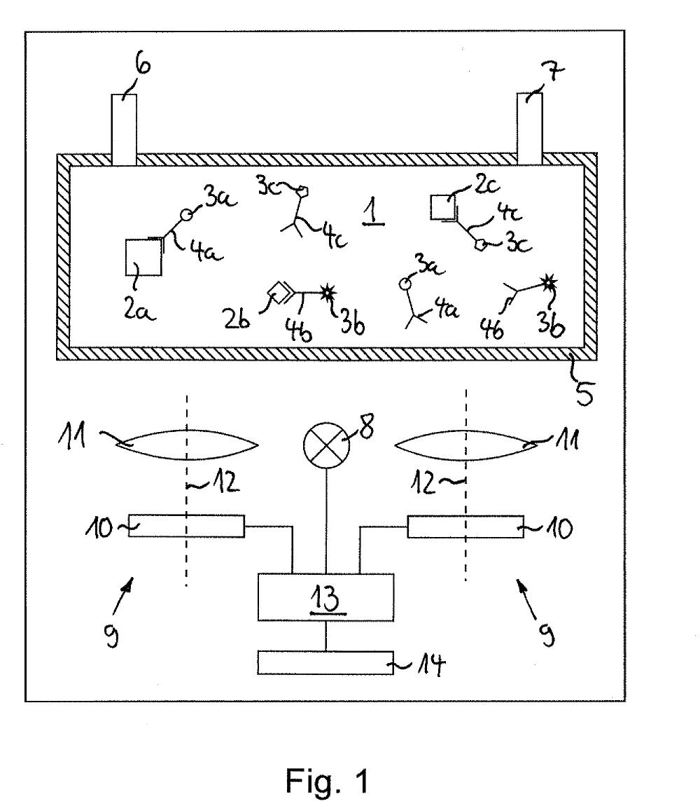

FIG. 1a cross section through a first example of an embodiment of a flow-through measurement chamber, and

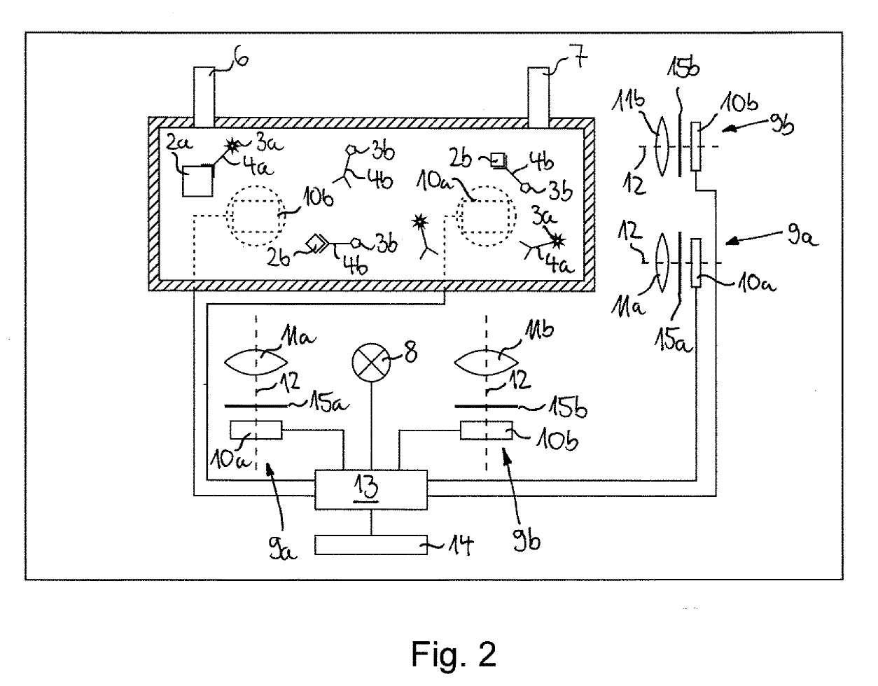

FIG. 2a cross section through a second example of an embodiment of a flow-through chamber.

In a method for the qualitative and quantitative detection of ligands 2a, 2b, 2c contained in a fluid 1, optical markers 3a, 3b, 3c of different marker types are placed in the fluid 1. Markers 3a, 3b, 3c are each linked to a detection antibody 4a, 4b, 4c that is binding-specific for a ligand 2a, 2b, 2c correlated with the respective marker type.

When markers 3a, 3b, 3c are irradiated with an optical excitation radiation, they each emit a luminescence radiation. The marker types are selected in such a way that the wavelengths of their luminescence radiations are distinguished from each other. In this way, for example, the 3a markers of a first marker type can be stimulated by the excitation radiation to emit green light, the 3b markers of a second marker type can be stimulated to emit blue light, and the markers 3c of a third marker type can be stimulated to emit red light.

After the complexes consisting of markers 3a, 3b, 3c and the detection antibodies 4a, 4b, 4c are mixed with the fluid 1, the mixture is placed in a measurement chamber 5 of a device shown in FIG. 1 that possesses a measurement apparatus for measuring the diffusion rate of markers 3a, 3b, 3c of the marker detection antibody ligand-complexes. The measurement chamber is a flow-through measurement chamber having an interior cavity, an inlet opening 6, and an outlet opening 7. The measurement chamber is temperature-controlled to a predetermined temperature.

The walls of the measurement chamber 5 are transparent to the excitation radiation and the luminescence radiations. In order to generate the excitation radiation, a light source 8, which projects light in the direction of the measurement chamber 5, is located at the measurement chamber 5. The light source 8 may be a light-emitting diode, for example.

As can be seen in FIG. 1, the measurement device used to measure the rate of diffusion has, adjacent to the measurement chamber 5, two cameras that each have an optical 2-dimensional sensor 10 that is sensitive to the luminescence radiations and that is not sensitive to the excitation radiation, for example a CCD sensor having pixels for red, green, and blue light, and an imaging optical system 11. The imaging optical system 11 reproduces the markers 3a, 3b, 3c that are located in the interior cavity of the measurement chamber 5 on the 2-dimensional sensor 10 that is assigned to the imaging optical system 11. The cameras are arranged with their optical axes 12 parallel to reach other, and are laterally separated from each other.

After the detection antibodies 4a, 4b, 4c, have been brought into contact with the fluid, a wait time must pass so that the detection antibodies 4a, 4b, 4c have a chance to bind to the ligands 2a, 2b, 2c. The fluid 1 including markers 3a, 3b, 3c contained in it is irradiated with the excitation radiation in order to induce markers 3a, 3b, 3c to emit their luminescence radiation. When this is done markers 3a, 3b, 3c are reproduced as light pixels on the two 2-dimensional sensors 10.

The 2-dimensional sensors 10 are connected to a control and evaluation device 13 that determines the positions of the individual markers 3a, 3b, 3c in three-dimensional space by means of triangulation from the position of the images of the markers 3a, 3b, 3c on the 2-dimensional sensors 10 and characteristic values for the position and arrangement of the cameras as well as their imaging characteristics. In this way, position measurement values for the individual markers 3a, 3b, 3c are determined cyclically with the aid of the camera 9. Based on the changes of the measured position values over time, measured values for the diffusion rate of markers 3a, 3b, 3c are determined. Since the diffusion rate of a particle in a fluid is dependent on the mass of the particle, markers 3a, 3b, 3c that are only bound to one detection antibody have a faster diffusion rate than markers that, in addition to being bound to a detection antibody, are also bound to a ligand 2a, 2b, 2c.

The diffusion rate measured values determined in this way are compared with reference value ranges that are stored in a data memory 14 that is connected to the control and evaluation device 13. Each reference value range is assigned to a particular ligand-antibody marker complex. If the diffusion rate measured values coincide with the respective reference value range, it is established that the marker 3a, 3b, 3c associated with the reference value range is contained in the fluid. The result of the comparison can be output by means of a display device that is connected to the control and evaluation device 13 and that is not specifically shown in the drawing.

The number of markers 3a, 3b, 3c whose diffusion rate coincides with the reference value range is also determined for each ligand type. With the aid of the quantity, the volume of the fluid 1, and the specified characteristic values, the concentration of the ligands 3a, 3b, 3c in the fluid 1 is determined for each ligand type.

In the example of the embodiment shown in FIG. 2, the measurement device used to measure the diffusion rate has three 2-dimensional sensors 10a, 10b, for each marker type. An imaging optical system 11a, 11b and an optical filter 15a, 15b that passes the luminescence radiation for the given marker type is assigned to each 2-dimensional sensor 10a, 10b. The 3-D cameras 9a, 9b that comprise the imaging optics 11a, 11b, the optical filter 15a, 15b, and the 2-dimensional sensor 10a, 10b, have their optical axes 12 arranged at right angles to each other. The 2-dimensional sensors 10a, 10b are connected to a common evaluation device 13, which, utilizing the images of the markers 3a, 3b on the 2-dimensional sensors 10a, lob, and characteristic values for the position and arrangements of the optical axes 12 and the imaging properties of the imaging optical systems 11a, 11b, determines three-dimensional measured position values for the individual markers 3a, 3b. As can be seen in the example of the embodiment shown in FIG. 1, measured values for the diffusion rate of the markers 3a, 3b, 3c are determined on the basis of the changes in the measured position values over time.

The evaluation device 13 is attached to a data memory 14 in which one reference value range for the diffusion rate is stored for each of the different ligand-antibody marker complexes. By comparing the measured diffusion rates with the respective reference ranges, it is determined whether the ligands 2a, 2b are present in the fluid.

It should also be noted that the ligands 2a, 2b, 2c of the various ligand types may differ from each other with respect to their mass and/or their size. However, the method may also be used to detect ligands of different ligand types if they have the same mass and/or the same size.

1. A method for detecting at least one ligand, that is suspected to be present in a fluid that is to be tested, in which at least one marker that is suitable for specifically binding to the ligands is placed in the fluid wherein at least one measured value for the diffusion rate of the marker and/or of a complex, consisting at least of the ligand and the marker bound to it is measured in the fluid, a reference value range is provided for the diffusion rate of the marker-ligand complex in the fluid, and the measured value is compared with the reference value range and the presence of the ligand in the fluid is deduced according to the result of this comparison.

2. The method of claim 1 wherein the marker is an optical marker, the fluid is irradiated with an excitation radiation, by which means the markers are excited to emit a luminescence radiation, the marker is reproduced on an optical 2-dimensional sensor that is sensitive to the luminescence radiation, and the marker diffusion rate is determined by measuring the movement velocity with which the image of the marker changes its position on the 2-dimensional sensor.

3. The method of claim 1, where the marker is an optical marker wherein the marker is selected in such a way that the spectrum of the luminescence radiation emitted by it is dependent on the binding of the marker to the ligand, the luminescence radiation emitted by the marker is measured and compared with a predetermined value range for at least one wavelength, and, depending on the results of this comparison, the presence of the ligand in the fluid is deduced.

4. The method of claim 2, wherein the optical marker is preferably reproduced with the aid of imaging optics on at least two, and preferably at least three optical 2-dimensional sensors whose measurement planes preferably extend transversely to each other, and which are sensitive to the luminescence radiation, the motion velocities with which the images of the marker change their position of the detection sensor are determined, and the diffusion rate of the marker is determined from the movement velocities and characteristic values for the relative position of the 2-dimensional sensors and preferably of the imaging optics.

5. The method of claim 2, wherein a plurality of similar markers are placed in the fluid, the number of markers whose diffusion rate coincides with the reference value range is determined, and the concentration of the marker in the fluid is determined from the number of markers.

6. The method of claim 2, wherein the fluid is irradiated with a planar beam of excitation radiation whose radiation plane preferably is parallel to the plane of the surface sensor.

7. The method of claim 2, wherein, in order to image ligands located in various areas of the fluid on the 2-dimensional sensor, the radiation beam is moved transverse to the plane of extension of the 2-dimensional sensor and/or tipped relative to said plane.

8. The method of claim 2, wherein at least two types of markers that are binding-specific for various ligands and that emit luminescence radiation at various wavelengths when irradiated with excitation radiation are placed in the fluid, the diffusion rates for the individual marker types are determined by measuring the rates of movement with which the images of the marker of the given marker type change their position on the 2-dimensional sensor for each of the wavelengths, and the measured values thus obtained for the diffusion rates are each compared with a reference value range assigned to one of the respective marker types and, in each case depending on the result of the comparison, the presence of the ligands that are binding-specific for the given marker type in the fluid is deduced.

9. The method of claim 1, wherein at least one ligand is provided by separating at least one substrand from the DNA and/or RNA molecule contained in the fluid and is replicated by means of a polymerase chain reaction.

10. The method of claim 1, wherein the fluid is heated and/or cooled to a predetermined temperature before the measured value for the diffusion rate of the marker is determined, and a measured value for the diffusion rate is determined, preferably at each of at least two different fluid temperatures.

11. The method of claim 1, wherein at least two types of markers that are binding-specific for different ligands and that differ from each other with respect to the extinction time of their luminescence radiation are placed in the fluids, the irradiation of the fluid with an excitation radiation is interrupted or ended and for the images of the individual markers on the 2-dimensional sensor in each case at least one measured value is determined for the extinction time, the images are assigned to a marker type by comparing these measured values with reference values, the diffusion rates for the individual marker types are determined by measuring the movement velocities with which the images of the markers of the given marker type change their position on the 2-dimensional sensor for each marker type, and the resulting measured values for the diffusion rates are each compared with a reference value range assigned to the given marker type and, depending on the result of the comparison the presence of the ligand that is binding-specific for the respective marker type is deduced in the fluid.

12. The method of claim 1, wherein the individual markers have a different mass.

13. An apparatus for detecting at least one ligand that can be specifically marked with a marker, said ligand being suspected of being president in a fluid that is to be tested, wherein the apparatus has a measuring device for measuring the diffusion rate that the marker and/or a complex comprising at least the ligand and the marker bound to it exhibit in the fluid, the device for preparing a reference value range for the diffusion rate of the marker-ligand complex has a reference value generator, and the measuring device and the reference value generator are connected to a comparison device in order to compare the measured diffusion rate with the reference value range.

14. The apparatus of claim 13 wherein said apparatus has a receiving area for the fluid, the measuring device has at least one optical 2-dimensional sensor that is sensitive to the luminescence radiation emitted by the marker and that is disposed in such a way relative to the receiving area that luminescence radiation emitted by at least one marker located in the receiving area is reproduced on the 2-dimensional sensor, and the 2-dimensional sensor is connected to an evaluation device that is designed in such a way that it is able to determine the diffusion rate from the changes in the measurement signal from the 2-dimensional sensor over time.

15. The apparatus of claim 13, wherein said apparatus has at least three of the optical 2-dimensional sensors and these 2-dimensional sensors are disposed with their planes of extension perpendicular to each other in order to measure the diffusion rate in three dimensions.

16. The apparatus of claim 13, wherein the receiving area for the fluid is enclosed by a measurement chamber, and the 2-dimensional sensor, of which there is at least one, and/or at least one light source is integrated into a wall of the measurement chamber in order to emit an excitation radiation for the markers.

17. The apparatus of claim 13, wherein the measurement device has at least two 2-dimensional sensors that are sensitive to various spectral ranges and/or have optical filters that allow optical radiation to pass in various spectral ranges and that are placed ahead of them.

18. The apparatus of claim 13, wherein said apparatus has a temperature control device for the receiving area.
